(19) United States
(12) Patent Application Publication
(10) Pub. No.: US 2012/0251630 A1
(43) Pub. Date: Oct. 4, 2012

(54) REMISSION THERAPY OF CANCER WITH ISOFLAVONOIDS

(75) Inventors: Ayesha B. Alvero, Stratford, CT (US); Daniel P. Gold, San Diego, CA (US); Gil G. Mor, Cheshire, CT (US)

(73) Assignee: MARSHALL EDWARDS, INC., San Diego, CA (US)

(21) Appl. No.: 13/434,665

(22) Filed: Mar. 29, 2012

Related U.S. Application Data
(60) Provisional application No. 61/469,066, filed on Mar. 29, 2011.

(51) Int. Cl. A61K 31/353; A61K 31/7068; C12N 5/095; A61K 31/4745; A61P 35/00; A61K 33/24; A61K 31/7048

(52) U.S. Cl. 424/649; 514/456; 514/49; 514/34; 514/283; 435/375

(57) ABSTRACT

Provided herein is a method of reducing incidences of cancer recurrence. The method involves administering to an individual in cancer remission an isoflavonoid. In specific instances, the treated individual is in remission from epithelial cancer, such as ovarian cancer or breast cancer.

### CROSS-REFERENCE

This application claims the benefit of U.S. Provisional Application No. 61/469,066, filed Mar. 29, 2011, which application is incorporated herein by reference.

### BACKGROUND OF THE INVENTION

Cancer is the leading cause of death worldwide.

### SUMMARY OF THE INVENTION

Provided herein is a method of reducing incidences of cancer recurrence. The method involves administering to an individual in cancer remission an isoflavonoid. In one embodiment of the invention, the treated individual is in remission from epithelial cancer, such as ovarian cancer or breast cancer.

Provided herein in some embodiments is a method of reducing incidences of cancer recurrence. The method involves administering to an individual in cancer remission an effective amount of a compound of formula (I):

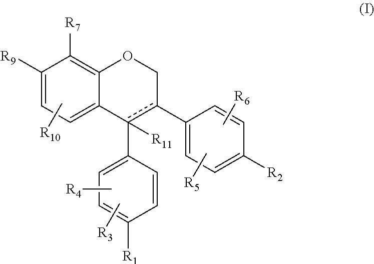

wherein
R₁, R₃ and R₄ are independently hydrogen, hydroxy, halo, haloalkyl, NR₁₂R₁₃, C₁₋₆ alkoxy, C₁₋₆ fluoroalkyl or C₁₋₆ alkyl;
R₂ and R₅ are independently hydrogen, hydroxy, halo, haloalkyl, NR₁₂R₁₃, C₁₋₆ alkoxy, C₁₋₆ fluoroalkyl or C₁₋₆ alkyl;
R₆ is hydrogen;
R₇ is hydrogen, haloalkyl, halo or C₁₋₆ alkyl;
R₉ is hydroxy or C₁₋₆ alkoxy;
R₁₀ is hydrogen;
the drawing “ [symbol] ” and R₁₁ together represent a double bond or the drawing “ [symbol] ” represents a single bond and R₁₁ is hydrogen or hydroxy;
R₁₂ and R₁₃ are independently hydrogen, C₁₋₆ alkyl or trialkyl silyl;

or salts or a derivative thereof; and wherein the compound of formula (I) reduces cancer cell proliferation.

In one embodiment, the method involves further administering to the individual an anti-cancer agent, wherein the combination of the compound of formula (I) and the anti-cancer agent reduces cancer cell proliferation. In some embodiments, the individual to be treated is in remission from an epithelial cancer selected from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma. In other embodiments, the anti-cancer agent is selected from cisplatin, carboplatin, paclitaxel, gemcitabine, doxorubicin, camptothecin, topotecan, taxol, and any combinations thereof.

In some embodiments, provided herein is a method of inhibiting loss of cancer remission comprising contacting an individual having cancer with an effective amount of formula (I). In one embodiment, the method involves further administering to the individual an anti-cancer agent, wherein the combination of the compound of formula (I) and the anti-cancer agent reduces cancer cell proliferation. In some embodiments, the individual to be treated is in remission from an epithelial cancer selected from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma. In other embodiments, the anti-cancer agent is selected from cisplatin, carboplatin, paclitaxel, gemcitabine, doxorubicin, camptothecin, topotecan, taxol, and any combinations thereof.

In other embodiments, provided herein is a method of inducing apoptosis in a cancer-related stem cell, the method comprising contacting said stem cell with a compound of formula (I). In one embodiment, the method involves further administering to the individual an anti-cancer agent, wherein the combination of the compound of formula (I) and the anti-cancer agent reduces cancer cell proliferation. In some embodiments, the individual to be treated is in remission from an epithelial cancer selected from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma. In other embodiments, the anti-cancer agent is selected from cisplatin, carboplatin, paclitaxel, gemcitabine, doxorubicin, camptothecin, topotecan, taxol, and any combinations thereof.

Provided herein in certain embodiments is a method of treating disease caused by cancer-related stem cells comprising administrating a therapeutically effective amount of a compound of formula (I). In one embodiment, the method involves further administering to the individual an anti-cancer agent, wherein the combination of the compound of formula (I) and the anti-cancer agent reduces cancer cell proliferation. In some embodiments, the individual to be treated is in remission from an epithelial cancer selected from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma. In other embodiments, the anti-cancer agent is selected from cisplatin, carboplatin, paclitaxel, gemcitabine, doxorubicin, camptothecin, topotecan, taxol, and any combinations thereof.

In some embodiments, provided herein is a method of treating radio-resistant epithelial cancer comprising administrating a therapeutically effective amount of a compound of formula (I). In one embodiment, the method involves further administering to the individual an anti-cancer agent, wherein the combination of the compound of formula (I) and the anti-cancer agent reduces cancer cell proliferation. In some embodiments, the individual to be treated is in remission from an epithelial cancer selected from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma. In other embodiments, the anti-cancer agent is selected from cisplatin, carboplatin, paclitaxel, gemcitabine, doxorubicin, camptothecin, topotecan, taxol, and any combinations thereof.

In some embodiments, provided herein is a method of treating chemo-resistant epithelial cancer comprising administrating a therapeutically effective amount of a compound of formula (I). In one embodiment, the method involves further administering to the individual an anti-cancer agent, wherein the combination of the compound of formula (I) and the anti-cancer agent reduces cancer cell proliferation. In some embodiments, the individual to be treated is in remission from an epithelial cancer selected from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma. In other embodiments, the anti-cancer agent is selected from cisplatin, carboplatin, paclitaxel, gemcitabine, doxorubicin, camptothecin, topotecan, taxol, and any combinations thereof.

### INCORPORATION BY REFERENCE

All publications and patent applications, including U.S. Pat. No. 7,601,855, U.S. Pat. No. 8,080,675, and PCT application number PCT/US2011/058815, mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. EOC stem cells were treated with 10 μg/ml compound 1 at designated times. Flow cytometry analysis (FIG. 1) illustrates the increased levels of mitochondrial superoxide using MitoSox dye.

FIG. 2 illustrates the levels of ATP and ADP measured at designated times using Apo-SENSOR ADP/ATP ratio assay kit. A decrease in ATP production is observed. (p<0.0001 compared to control)

FIG. 3. Western blot analysis illustrates a decrease in Cox-IV and pS6 kinase, and activation of ERK and AMPKα1 (C).

FIG. 4. EOC stem cells were treated with compound 1 in the presence or absence of the ROS scavenger, MnTBAP. Western blot analysis (FIG. 2) illustrates the effect of MnTBAP pretreatment on mitochondrial ERK, pS6 and β-actin activation. MnTBAP inhibited compound 1-induced activation of mitochondrial ERK, but not compound 1 induced decrease in pS6 kinase. Compound 1-induced ROS production leads to ERK activation and loss of mitochondrial membrane potential (MMP).

FIG. 5. EOC stem cells were treated with compound 1 in the presence or absence of the MEK inhibitor, U0126. Flow cytometry analysis using JC1 dye illustrates the effect on mitochondrial membrane potential (MMP). FIG. 5 illustrates that inhibition of ERK with U0126 is able to reverse the effect of compound 1 on MMP.

FIG. 6. Western blot analysis using mitochondrial fractions illustrates the effect on mitochondrial Bax. U0126 inhibited compound 1-induced loss of mitochondrial membrane potential and mitochondrial translocation of Bax (member of the proapoptotic Bcl₂ family).

FIG. 7. Compound 1-induced loss of ATP leads to mTor inhibition. EOC stem cells were treated with compound 1 in the presence of 10% FBS. FIG. 7 illustrates the levels of ATP and ADP measured at designated times using ApoS-SENSOR ADP/ATP ratio assay kit. # p<0.001 compared to compound 1 alone.

FIG. 8. Western blot analysis illustrates that treatment of ovarian cancer stem cells with compound 1 in the presence of FBS was able to prevent compound 1 induced decrease in pS6 kinase.

FIG. 9. Proposed model for compound 1-induced cell death in EOC stem cells. By targeting the mitochondria, compound 1 activates two independent cell death pathways. Degradation of Cox-IV leads to ATP loss and increase mitochondrial ROS. ATP loss leads to inhibition of mTOR pathway and autophagic cell death. ROS activates the ERK/Bax axis leading to loss of mitochondrial membrane potential and EndoGdependent DNA fragmentation.

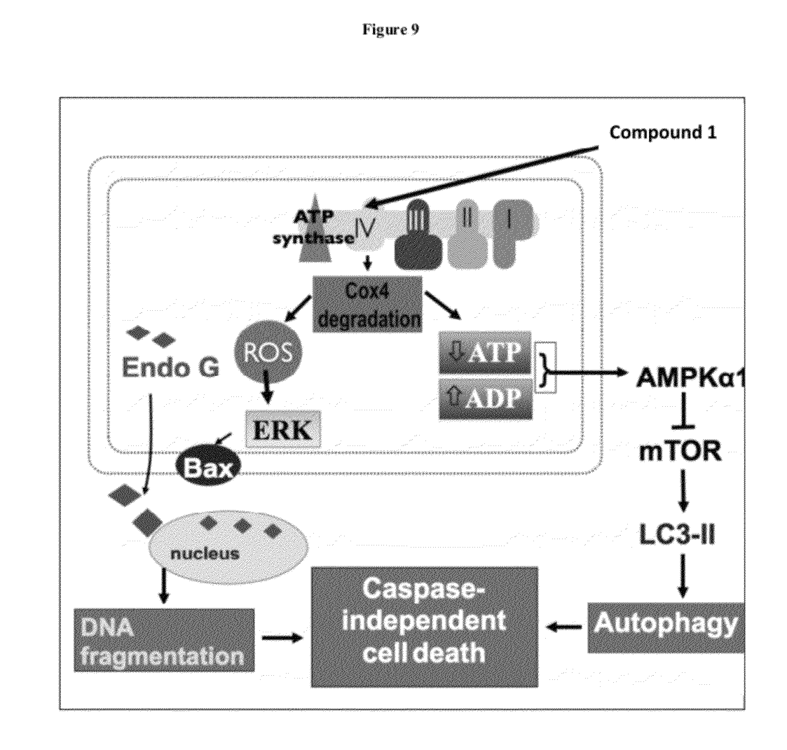

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Definitions

Unless otherwise noted, terminology used herein should be given its normal meaning as understood by one of skill in the art.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The terms “C₁-C₃-alkyl” and “C₁-C₆-alkyl” as used herein refer to saturated, straight- or branched-chain hydrocarbon radicals derived from a hydrocarbon moiety containing between one and three, one and six, and one and twelve carbon atoms, respectively, by removal of a single hydrogen atom. Examples of C₁-C₃-alkyl radicals include methyl, ethyl, propyl and isopropyl. Examples of C₁-C₆-alkyl radicals include, but not limited to, methyl, ethyl, propyl, isopropyl, n-butyl, tert-butyl, neopentyl and n-hexyl.

The term “alkoxy” as used herein, alone or in combination, refers to an alkyl ether radical, —O-alkyl, including the groups —O-aliphatic and —O-carbocyclyl, wherein the alkyl, aliphatic and carbocyclyl groups may be optionally substituted, and wherein the terms alkyl, aliphatic and carbocyclyl are as defined herein. Non-limiting examples of alkoxy radicals include methoxy, ethoxy, n-propoxy, isopropoxy, n-butoxy, iso-butoxy, sec-butoxy, tert-butoxy and the like.

The terms “C₁-C₃-alkoxy”, “C₁-C₆-alkoxy” as used herein refers to the C₁-C₃-alkyl group and C₁-C₆-alkyl group, as previously defined, attached to the parent molecular moiety through an oxygen atom. Examples of C₁-C₆-alkoxy radicals include, but not limited to, methoxy, ethoxy, propoxy, isopropoxy, n-butoxy, tert-butoxy, neopentoxy and n-hexoxy.

The term “halo” and “halogen” as used herein refer to an atom selected from fluoro, chloro, bromo and iodo.

The term “haloalkyl” includes “alkyl” wherein one or more such as 1, 2, 3, 4, or 5 of the hydrogens have been replaced by a halo atom. The haloalkyl may be straight chain or branched chain “alkyl” unit. Non-limiting examples include —CH₂F, —CHF₂, —CF₃, —CH₂CH₂F, —CH₂CHF₂, —CH₂CF₃, —CF₂CH₂F, —CF₂CHF₂, —CF₂CF₃, —CH₂Cl, —CHCl₂, —CCl₃, —CH₂Br, —CHBr₂, and —CBr₃.

The term “fluoroalkyl” includes “alkyl” wherein one or more such as 1, 2, 3, 4, or 5 of the hydrogens have been replaced by fluoro. The fluoroalkyl may be straight chain or branched chain “alkyl” unit. Preferred fluoroalkyl groups include trifluoromethyl and pentafluoroethyl.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. For example, S. M. Berge, et al. describes pharmaceutically acceptable salts in detail in J. Pharmaceutical Sciences, 66: 1-19 (1977), incorporated herein by reference for this purpose. The salts are prepared in situ during the final isolation and purification of the compounds described herein, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

It should be understood that a reference to a salt includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

As used herein, the term “effective amount” when used in reference to reducing the severity of a proliferative disease, such as cancer, means an amount of a compound of formula (I) and/or an anti-cancer agent administered to an individual required to effect a decrease in the extent, amount or rate of spread of a neoplastic condition or pathology. When used in reference to reducing cancer recurrence, the term means an amount of a compound of formula (I) and/or an anti-cancer agent administered to an individual required to reduce cancer recurrence or risk of cancer recurrence. The amount of a compound of formula (I) and/or an anti-cancer agent required to be effective will depend, for example, on the type of anti-cancer agent administered and the pathological condition to be treated, as well as the weight and physiological condition of the individual, and previous or concurrent therapies. An amount considered as an effective amount for a particular application of a compound of formula (I) and an anti-cancer agent will be known or can be determined by those skilled in the art, using the teachings and guidance provided herein. One skilled in the art will recognize that the condition of the patient can be monitored throughout the course of therapy and that the amount of the modulating compound that is administered can be adjusted according to the individual's response to therapy.

The term “patient”, “subject” or “individual” are used interchangeably. As used herein, they refer to individuals suffering from a disorder, and the like, encompasses mammals and non-mammals None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The terms “treat”, “treating” or “treatment”, as used herein, include alleviating, abating or ameliorating a disease or condition or one or more symptoms thereof, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease).

The term “carrier” as used herein, refers to relatively nontoxic chemical compounds or agents that facilitate the incorporation of a compound into cells or tissues.

### Methods

It has been hypothesized that tumor stem cells are responsible for maintaining the malignant potential of a tumor, and may serve as an underlying cause of tumor recurrence. Current treatment strategies may fail to target the drug-resistant subpopulation, which may explain the initial therapeutic response of the majority of tumor cells followed by later recurrence.

In some embodiments, cancer stem cells demonstrate one or more characteristics that include: 1) evasion of apoptosis, 2) unlimited replicative ability, 3) potential for tissue remodeling with invasion, and 4) formation of distant metastases.

Some embodiments provided herein describe compounds that create a state of cellular starvation and activate two noncanonical pathways to induce cell death in cancer stem cells. In some embodiments, the compounds described herein activate the 5′AMP kinase (AMPK)-mTOR pathway. In some embodiments, the compounds described herein active the extracellular signal-regulated kinase (ERK)-Bax pathway. In some embodiments, the compounds described herein inhibit mitochondrial function in cancer stem cells. In certain embodiments, the compounds described herein lead to a loss of mitochondrial membrane potential. In other embodiments, the compounds described herein induce EndoG-dependent DNA fragmentation. In some embodiments, the compounds described herein induce degradation of Cox-1 and IV, leading to ATP loss and an increase of mitochondrial reactive oxygen species (ROS). In further or additional embodiments, the ATP loss leads to inhibition of mTOR pathway. In some embodiments, the compounds described herein induce death in apoptosis-resistant cancer stem cells.

In some embodiments, an individual in remission from cancer is treated according to any method described herein to reduce the risk of cancer recurrence. As used herein, the term “recurrence” means growth or neoplastic or cancerous cells after a tumor or other cancerous condition has been successfully treated, such as by surgical or chemically-induced removal or disintegration of cancerous cells. In certain embodiments, recurrence involves dissemination of cancerous cells into local or distant tissues and organs with respect to the primary cancer.

Provided herein in some embodiments are methods for reducing the severity of a proliferative disorder and for reducing cancer recurrence, administration of a compound described herein or the combination of a compound described herein and an anti-cancer agent reduces cellular proliferation. As used herein, the term “reduces” when used in reference to cellular proliferation means affecting a decrease in the extent, amount or rate of cell growth.

In other embodiments, provided herein is a method of inducing apoptosis in a cancer-related stem cell, the method comprising contacting said stem cell with a compound of formula I, II or III. In one embodiment, the method involves further administering to the individual an additional anti-cancer agent, wherein the combination of the compound of formula I, II or III and the anti-cancer agent reduces cancer cell proliferation.

Provided herein in certain embodiments is a method of treating a disease caused by cancer-related stem cells comprising administrating a therapeutically effective amount of a compound of formula I, II, or III. In certain embodiments, the method involves further administering to the individual an additional anti-cancer agent, wherein the combination of the compound of formula I, II or III and the anti-cancer agent reduces cancer cell proliferation. In some embodiments, the disease caused by cancer-related stem cells is recurrent cancer. In some embodiments, the recurrent cancer is resistant to one or more anti-cancer agents (e.g., cisplatin, carboplatin, taxol, etc.). In certain embodiments, the recurrent cancer is recurrent breast cancer and recurrent ovarian cancer.

In some embodiments, administration of compound of formula I, II, or III or the combination of a compound of formula I, II, or III and one or more anti-cancer agents reduces cancer cell proliferation in order to reduce cancer recurrence in an individual in cancer remission. Accordingly, the invention provides a method of reducing cancer recurrence. The method involves administering to an individual in cancer remission an effective amount of a compound of formula I, II or III, wherein a compound of formula I, II, or III reduces cancer stem cell proliferation.

Any of the method described herein, in some embodiments, further comprise administering cancer therapy to the individual or patient. In certain embodiments, the cancer therapy is, by way of non-limiting example, at least one anti-cancer agent (e.g., chemotherapeutic agent), radiation therapy, or surgery. In some embodiments, a combination of (1) administration of an effective amount of a compound described herein and (2) 1 to 3 therapies selected from the group consisting of (i) administration of an effective amount of an additional anticancer agents, (ii) administration of an effective amount of hormonal therapeutic agents and (iii) non-drug therapy prevents and/or treats cancer more effectively.

An anti-cancer agent includes but is not limited to a chemotherapeutic agent, immunotherapeutic agent, a pharmaceutical agent that inhibits the action of cell growth factor and a receptor thereof and the like. Among the chemotherapeutic agents that are optionally employed, by way of non-limiting example, are cisplatin, carboplatin, paclitaxel, gemcitabine or doxorubicin. Further, non-limiting examples of chemotherapeutic agents include alkylating agents, antimetabolites, anticancer antibiotics, plant-derived anticancer agents, and the like.

Alkylating agents include but are not limited to nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, sodium estramustine phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin, and the like.

Antimetabolites include but are not limited to mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emitefur, and the like), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine and the like.

Anticancer antibiotics include but are not limited to actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, and the like.

Plant-derived anticancer agents include but are not limited to etoposide, etoposide phosphate, vinblastine sulfate, vincristine sulfate, vindesine sulfate, teniposide, paclitaxel, docetaxel, vinorelbine, and the like.

Immunotherapeutic agents include but are not limited to picibanil, krestin, sizofuran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, and the like.

Non-limiting examples of a cell growth factor in pharmaceutical agents that inhibit the action of cell growth factors or cell growth factor receptors include any substances that promote cell proliferation, which are normally peptides having a molecular weight of not more than 20,000 that are capable of exhibiting their activity at low concentrations by binding to a receptor, including (1) EGF (epidermal growth factor) or substances possessing substantially the same activity as it [e.g., EGF, heregulin, and the like], (2) insulin or substances possessing substantially the same activity as it [e.g., insulin, IGF (insulin-like growth factor)-1, IGF-2, and the like], (3) FGF (fibroblast growth factor) or substances possessing substantially the same activity as it [e.g., acidic FGF, basic FGF, KGF (keratinocyte growth factor), FGF-10, and the like], (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), VEGF (vascular endothelial growth factor), and the like], and the like.

Cell growth factor receptors include but are not limited to any receptors capable of binding to the aforementioned cell growth factors, including EGF receptor, heregulin receptor (HER2), insulin receptor, IGF receptor, FGF receptor-1 or FGF receptor-2, and the like.

Pharmaceutical agent that inhibits the action of cell growth factor include but are not limited to HER2 antibody (e.g., trastuzumab), imatinib mesylate, ZD1839 or EGFR antibody (e.g., cetuximab), antibody to VEGF (e.g., bevacizumab), VEGFR antibody, VEGFR inhibitor, and EGFR inhibitor (e.g., erlotinib).

In addition to the aforementioned drugs, other anti-cancer agents include but are not limited to L-asparaginase, aceglatone, procarbazine hydrochloride, protoporphyrin-cobalt complex salt, mercuric hematoporphyrin-sodium, topoisomerase I inhibitors (e.g., irinotecan, topotecan, and the like), topoisomerase II inhibitors (e.g., sobuzoxane, and the like), differentiation inducers (e.g., retinoid, vitamin D, and the like), angiogenesis inhibitors (e.g., thalidomide, SU11248, and the like), α-blockers (e.g., tamsulosin hydrochloride, naftopidil, urapidil, alfuzosin, terazosin, prazosin, silodosin, and the like) serine/threonine kinase inhibitor, endothelin receptor antagonist (e.g., atrasentan, and the like), proteasome inhibitor (e.g., bortezomib, and the like), Hsp 90 inhibitor (e.g., 17-AAG, and the like), spironolactone, minoxidil, 11α-hydroxyprogesterone, bone resorption inhibiting/metastasis suppressing agent (e.g., zoledronic acid, alendronic acid, pamidronic acid, etidronic acid, ibandronic acid, clodronic acid) and the like.

Non-limiting examples of hormonal therapeutic agents include fosfestrol, diethylstylbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, Tadenan, mepartricin, raloxifene, ormeloxifene, levormeloxifene, anti-estrogens (e.g., tamoxifen citrate, toremifene citrate, and the like), ER down-regulator (e.g., fulvestrant and the like), human menopausal gonadotrophin, follicle stimulating hormone, pill preparations, mepitiostane, testrolactone, aminoglutethimide, LH-RH agonists (e.g., goserelin acetate, buserelin, leuprorelin, and the like), droloxifene, epitiostanol, ethinylestradiol sulfonate, aromatase inhibitors (e.g., fadrozole hydrochloride, anastrozole, retrozole, exemestane, vorozole, formestane, and the like), anti-androgens (e.g., flutamide, bicartamide, nilutamide, and the like), 5α-reductase inhibitors (e.g., finasteride, dutasteride, epristeride, and the like), adrenocorticohormone drugs (e.g., dexamethasone, prednisolone, betamethasone, triamcinolone, and the like), androgen synthesis inhibitors (e.g., abiraterone, and the like), and retinoid and drugs that retard retinoid metabolism (e.g., liarozole, and the like), etc. and LH-RH agonists (e.g., goserelin acetate, buserelin, leuprorelin).

The non-drug therapy is exemplified by surgery, radiotherapy, gene therapy, thermotherapy, cryotherapy, laser cauterization, and the like, and any combinations thereof.

When a compound (i.e., isoflavonoid derivative) of Formula I, II, or III and a concomitant drug are used in combination, the administration time of the isoflavonoid derivative and the concomitant drug is not restricted. In some embodiments, the isoflavonoid derivative and the concomitant drug are administered to an individual simultaneously. In other embodiments, the isoflavonoid derivative and the concomitant drug are administered at staggered times.

In some embodiments, the cancer is selected from the group consisting of bladder cancer, breast cancer, metastatic breast cancer, metastatic HER2-negative breast cancer, colon cancer, rectal cancer, metastatic colorectal cancer, endometrial cancer, cervical cancer, uterine cancer, ovarian cancer, kidney cancer, liver cancer, leukemia, lung cancer (both small cell and non-small cell), squamous non-small cell lung cancer, non-squamous non-small cell lung cancer, melanoma, non-Hodgkin lymphoma, pancreatic cancer, testicular cancer, prostate cancer, thyroid cancer, sarcoma (including osteosarcoma), esophageal cancer, gastric cancer, head and neck cancer, lung cancer melanoma, myeloma, neuroblastoma, glioblastoma, and cancers of the brain. In some embodiments, the cancer is selected from, by way of non-limiting example, human breast, prostate, ovarian, pancreatic, or cervical cancer. In certain specific embodiments, the cancer is human breast cancer or ovarian cancer.

### Compounds

Some embodiments of the present invention describe a pharmaceutical composition comprising a compound (i.e., isoflavonoid derivative) of general formula I:

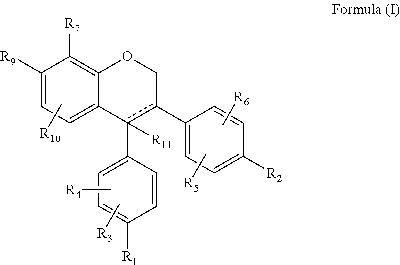

wherein
R₁ is hydrogen, hydroxy, halo, NR₁₄R₁₅, C₃₋₆cycloalkyl, C₁₋₆alkoxy, C₁₋₆haloalkyl, C₂₋₆alkenyl, COOR₁₂, COR₁₃, (O)ₙC₁₋₄alkyleneNR₁₄R₁₅ or C₁₋₆alkyl optionally substituted by one or more hydroxy, chloro, bromo, iodo or NR₁₄R₁₅ groups;
R₂, R₃, R₄, R₅, R₆, R₉, and R₁₀ are independently hydrogen, hydroxy, halo, NR₁₄R₁₅, C₃₋₆cycloalkyl, C₁₋₆alkoxy, C₁₋₆haloalkyl, C₂₋₆alkenyl, COOR₁₂, COR₁₃, or C₁₋₆alkyl optionally substituted by one or more hydroxy, chloro, bromo, iodo or NR₁₄R₁₅ groups;
R₇ is hydrogen, hydroxy, halo, NR₁₄R₁₅, C₃₋₆cycloalkyl, C₁₋₆alkoxy, C₂₋₆alkenyl, C₁₋₆haloalkyl or C₁₋₆alkyl optionally substituted by one or more hydroxy, chloro, bromo, iodo or NR₁₄R₁₅ groups;
the drawing [symbol] and R₂ together represent a double bond or the drawing [symbol] represents a single bond and R₁₁ is hydrogen, hydroxy, NR₁₄R₁₅, C₁₋₃alkoxy, C₁₋₃fluoroalkyl, halo or C₁₋₃alkyl optionally substituted by one or more hydroxy, chloro, bromo, iodo or NR₁₄R₁₅ groups;
R₁₁ and R₁₂ are independently hydrogen, C₁₋₆alkyl, C₃₋₆cycloalkyl, or trialkyl silyl;
R₁₃ is hydrogen, C₁₋₆alkyl, C₃₋₆cycloalkyl or NR₁₄R₁₅;
n represents 0 or 1; and
R₁₄ and R₁₅ independently represent hydrogen or C₁₋₆alkyl or NR₁₄R₁₅ when taken together represents a 5 or 6 membered heteroaromatic or heterocyclic,
or a pharmaceutically acceptable salt thereof.

In some embodiments, the pharmaceutical composition comprises a compound (i.e., isoflavonoid derivative) of formula II:

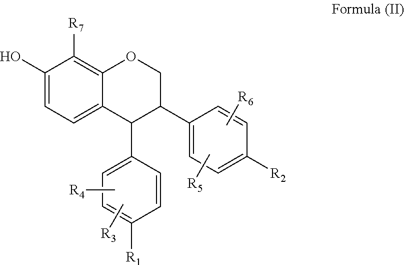

R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl; and
R₇ is hydrogen, alkyl, halo or haloalkyl;
or a pharmaceutically acceptable salt thereof.

Some embodiments provided herein describe a compound of Formula II that has a structure of Formula (II-a) or (II-b):

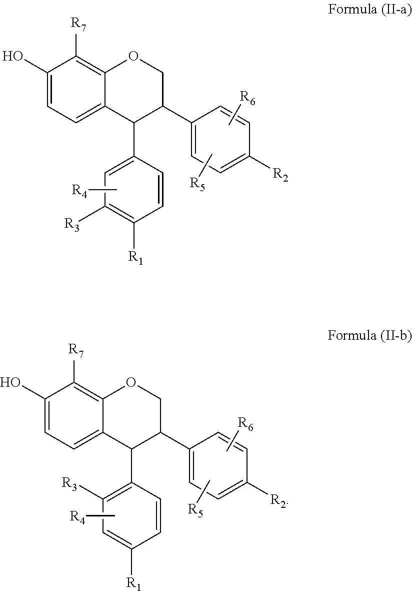

In some embodiments, R₁ is hydroxy. In other embodiments, R₁ is C₁-C₆alkoxy. In further or additional embodiments, R₁ is C₁-C₃alkoxy. In other embodiments, R₁ is C₁-C₂alkoxy. In specific embodiments, R₁ is methoxy. In specific embodiments, R₁ is ethoxy. In specific embodiments, R₁ is propoxy. In specific embodiments, R₁ is iso-propoxy. In specific embodiments, R₁ is butoxy. In specific embodiments, R₁ is iso-butoxy. In specific embodiments, R₁ is sec-butoxy. In specific embodiments, R₁ is tert-butoxy. In specific embodiments, R₁ is pentyloxy. In specific embodiments, R₁ is hexyloxy. In further or alternative embodiments, R₁ is fluoro. In other embodiments, R₁ is chloro. In other embodiments, R₁ is iodo. In other embodiments, R₁ is bromo. In other embodiments, R₁ is haloalkyl. In other embodiments, R₁ is haloC₁₋₆alkyl. In other embodiments, R₁ is haloC₁₋₃alkyl. In other embodiments, R₁ is haloC₁₋₂alkyl. In specific embodiments, R₁ is monofluoromethyl. In specific embodiments, R₁ is difluoromethyl. In specific embodiments, R₁ is trifluoromethyl.

In further or additional embodiments, R₂ is hydroxy. In some embodiments, R₂ is C₁-C₆alkoxy. In further or additional embodiments, R₂ is C₁-C₃alkoxy. In further or additional embodiments, R₂ is C₁-C₂alkoxy. In specific embodiments, R₂ is methoxy. In specific embodiments, R₂ is ethoxy. In specific embodiments, R₂ is propoxy. In specific embodiments, R₂ is iso-propoxy. In specific embodiments, R₂ is butoxy. In specific embodiments, R₂ is iso-butoxy. In specific embodiments, R₂ is sec-butoxy. In specific embodiments, R₂ is tert-butoxy. In specific embodiments, R₂ is pentyloxy. In specific embodiments, R₂ is hexyloxy.

In some embodiments, compounds of the general formula (II) have the substituents R₁, R₃, and R₄ distributed as shown below:

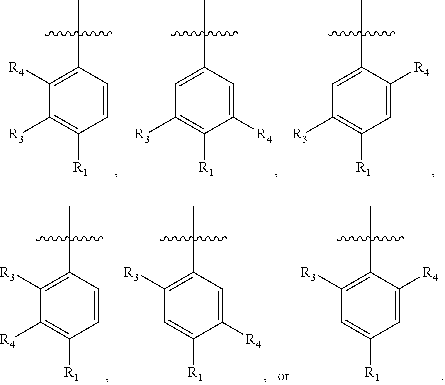

In some embodiments, R₃ is hydrogen. In further or additional embodiments, R₃ is C₁-C₆alkyl. In other embodiments, R₃ is C₁-C₃alkyl. In other embodiments, R₃ is C₁-C₂alkyl. In specific embodiments, R₃ is methyl. In specific embodiments, R₃ is ethyl. In specific embodiments, R₃ is propyl. In specific embodiments, R₃ is iso-propyl. In specific embodiments, R₃ is butyl. In specific embodiments, R₃ is iso-butyl. In specific embodiments, R₃ is sec-butyl. In specific embodiments, R₃ is tert-butyl. In specific embodiments, R₃ is pentyl. In specific embodiments, R₃ is hexyl. In further or alternative embodiments, R₃ is fluoro. In other embodiments, R₃ is chloro. In other embodiments, R₃ is iodo. In other embodiments, R₃ is bromo. In other embodiments, R₃ is haloalkyl. In other embodiments, R₃ is haloC₁₋₆alkyl. In other embodiments, R₃ is haloC₁₋₃alkyl. In other embodiments, R₃ is haloC₁₋₂alkyl. In specific embodiments, R₃ is monofluoromethyl. In specific embodiments, R₃ is difluoromethyl. In specific embodiments, R₃ is trifluoromethyl. In some embodiments, R₃ is hydrogen, halo or alkyl.

In further or additional embodiments, R₄ is hydrogen. In further or alternative embodiments, R₄ is halo. In specific embodiments, R₄ is fluoro. In other embodiments, R₄ is haloalkyl. In other embodiments, R₄ is haloC₁₋₆alkyl. In other embodiments, R₄ is haloC₁₋₃alkyl. In other embodiments, R₄ is haloC₁₋₂alkyl. In specific embodiments, R₄ is monofluoromethyl. In specific embodiments, R₄ is difluoromethyl. In specific embodiments, R₄ is trifluoromethyl. In further or alternative embodiments, R₄ is C₁-C₆alkyl. In other embodiments, R₄ is C₁-C₃alkyl. In other embodiments, R₄ is C₁-C₂alkyl. In specific embodiments, R₄ is methyl. In specific embodiments, R₄ is ethyl. In specific embodiments, R₄ is propyl. In specific embodiments, R₄ is iso-propyl.

Some embodiments provided herein describe a compound of formula II wherein R₅ and R₆ are hydrogen. In specific embodiments, R₅ is hydrogen. In other specific embodiments, R₆ is hydrogen.

In other embodiments, R₅ is alkyl. In other embodiments, R₅ is C₁-C₆alkyl. In other embodiments, R₅ is C₁-C₃alkyl. In other embodiments, R₅ is C₁-C₂alkyl. In specific embodiments, R₅ is methyl. In specific embodiments, R₅ is ethyl. In specific embodiments, R₅ is propyl. In specific embodiments, R₅ is iso-propyl. In other embodiments, R₅ is halo. In other embodiments, R₅ is fluoro. In other embodiments, R₅ is bromo. In other embodiments, R₅ is chloro. In other embodiments, R₅ is iodo. In other embodiments, R₅ is haloalkyl. In other embodiments, R₅ is haloC₁₋₆alkyl. In other embodiments, R₅ is haloC₁₋₃alkyl. In other embodiments, R₅ is haloC₁₋₂alkyl. In specific embodiments, R₅ is monofluoromethyl. In specific embodiments, R₅ is difluoromethyl. In specific embodiments, R₅ is trifluoromethyl.

In still further or alternative embodiments, R₆ is alkyl, haloalkyl or halo. In other embodiments, R₆ is alkyl. In other embodiments, R₆ is C₁-C₆alkyl. In other embodiments, R₆ is C₁-C₃alkyl. In other embodiments, R₆ is C₁-C₂alkyl. In specific embodiments, R₆ is methyl. In specific embodiments, R₆ is ethyl. In specific embodiments, R₆ is propyl. In specific embodiments, R₆ is iso-propyl. In other embodiments, R₆ is halo. In other embodiments, R₆ is fluoro. In other embodiments, R₆ is bromo. In other embodiments, R₆ is chloro. In other embodiments, R₆ is iodo. In other embodiments, R₆ is haloalkyl. In other embodiments, R₆ is haloC₁₋₆alkyl. In other embodiments, R₆ is haloC₁₋₃alkyl. In other embodiments, R₆ is haloC₁₋₂alkyl. In specific embodiments, R₆ is monofluoromethyl. In specific embodiments, R₆ is difluoromethyl. In specific embodiments, R₆ is trifluoromethyl.

In some embodiments, R₇ is hydrogen. In some embodiments, R₇ is C₁-C₆alkyl. In other embodiments, R₇ is C₁-C₃alkyl. In other embodiments, R₇ is C₁-C₂alkyl. In specific embodiments, R₇ is methyl. In specific embodiments, R₇ is ethyl. In specific embodiments, R₇ is propyl. In specific embodiments, R₇ is isopropyl. In alternative embodiments, R₇ is hydrogen.

Provided herein, in some embodiments, is a pharmaceutical composition comprising a compound (i.e., isoflavonoid derivative) of formula III:

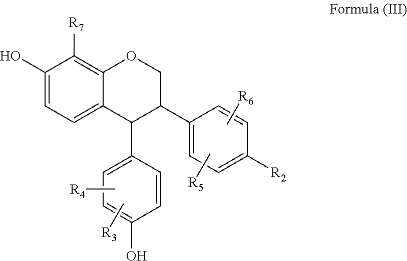

wherein
R₂ is hydroxy or alkoxy;
R₃, R₄, R₅, and R₆ are independently hydrogen, hydroxy, alkoxy, halo, haloalkyl, or alkyl; and
R₇ is alkyl or hydrogen;
or a pharmaceutically acceptable salt thereof.

Some embodiments provided herein describe a compound of Formula III that has a structure of Formula (III-a) or (III-b):

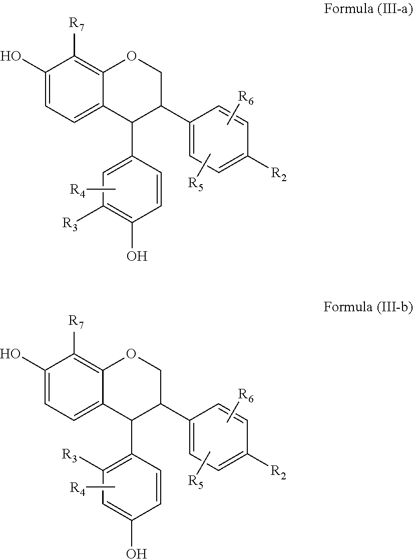

In some embodiments, R₂ is hydroxy. In other embodiments, R₂ is C₁-C₆alkoxy. In further or additional embodiments, R₂ is C₁-C₃alkoxy. In specific embodiments, R₂ is methoxy, ethoxy, propoxy, iso-propoxy, butoxy, iso-butoxy, sec-butoxy, tert-butoxy, pentyloxy or hexyloxy. In specific embodiments, R₂ is methoxy.

In further or additional embodiments, R₃ is C₁-C₆alkyl. In other embodiments, R₃ is C₁-C₃alkyl. In other embodiments, R₃ is C₁-C₂alkyl. In other embodiments, R₃ is methyl. In other embodiments, R₃ is ethyl. In other embodiments, R₃ is propyl. In other embodiments, R₃ is iso-propyl. In other embodiments, R₃ is butyl. In other embodiments, R₃ is iso-butyl. In other embodiments, R₃ is sec-butyl. In other embodiments, R₃ is tert-butyl. In other embodiments, R₃ is pentyl. In other embodiments, R₃ is hexyl. In alternative embodiments, R₃ is hydrogen. In other embodiments, R₃ is halo. In other embodiments, R₃ is fluoro. In other embodiments, R₃ is chloro. In other embodiments, R₃ is bromo. In other embodiments, R₃ is haloalkyl. In other embodiments, R₃ is haloC₁₋₆alkyl. In other embodiments, R₃ is haloC₁₋₃alkyl. In other embodiments, R₃ is haloC₁₋₂alkyl. In specific embodiments, R₃ is monofluoromethyl. In specific embodiments, R₃ is difluoromethyl. In specific embodiments, R₃ is trifluoromethyl.

In further or additional embodiments, R₄ is hydrogen. In further or alternative embodiments, R₄ is halo. In specific embodiments, R₄ is fluoro. In specific embodiments, R₄ is chloro. In specific embodiments, R₄ is bromo. In other embodiments, R₄ is haloalkyl. In other embodiments, R₄ is haloC₁₋₆alkyl. In other embodiments, R₄ is haloC₁₋₃alkyl. In other embodiments, R₄ is haloC₁₋₂alkyl. In specific embodiments, R₄ is monofluoromethyl. In specific embodiments, R₄ is difluoromethyl. In specific embodiments, R₄ is trifluoromethyl. In other embodiments, R₄ is C₁-C₆alkyl. In other embodiments, R₄ is C₁-C₃alkyl. In other embodiments, R₄ is C₁-C₂alkyl. In other embodiments, R₄ is methyl. In other embodiments, R₄ is ethyl. In other embodiments, R₄ is propyl. In other embodiments, R₄ is iso-propyl.

In some embodiments, R₇ is C₁-C₆alkyl. In other embodiments, R₇ is C₁-C₃alkyl. In other embodiments, R₇ is C₁-C₂alkyl. In specific embodiments, R₇ is methyl. In other embodiments, R₇ is ethyl. In other embodiments, R₇ is propyl. In other embodiments, R₇ is iso-propyl. In other embodiments, R₇ is butyl. In other embodiments, R₇ is iso-butyl. In other embodiments, R₇ is sec-butyl. In other embodiments, R₇ is tert-butyl. In other embodiments, R₇ is pentyl. In other embodiments, R₇ is hexyl.

In some embodiments, compounds of the general Formula III have the substituents R₃ and R₄ distributed as shown below:

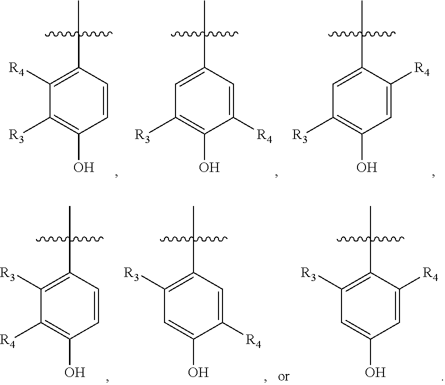

In some embodiments, compounds of the general Formula I, II, or III have the substituents R₂, R₅, and R₆ distributed as shown below:

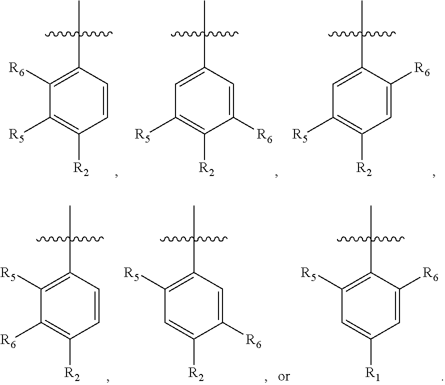

In some embodiments, the pharmaceutical composition comprises a compound (i.e., isoflavonoid derivative) of formula I:

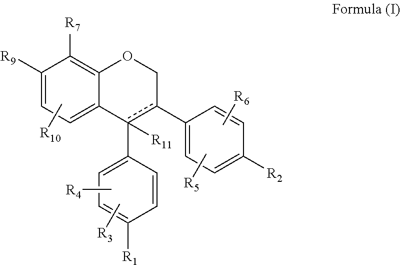

wherein
R₁, R₃ and R₄ are independently hydrogen, hydroxy, halo, NR₁₂R₁₃, C₁₋₆ alkoxy, C₁₋₆ fluoroalkyl or C₁₋₆ alkyl;
R₂ and R₅ are independently hydrogen, hydroxy, halo, NR₁₂R₁₃, C₁₋₆ alkoxy, C₁₋₆ fluoroalkyl or C₁₋₆ alkyl; and
R₆ is hydrogen;
R₇ is halo or C₁₋₆ alkyl;
R₉ is hydroxy or C₁₋₆ alkoxy;
R₁₀ is hydrogen;
the drawing “ [symbol] ” and R₁₁ together represent a double bond or the drawing “ [symbol] ” represents a single bond and R₁₁ is hydrogen or hydroxy,
R₁₂ and R₁₃ are independently hydrogen, C₁₋₆ alkyl or trialkyl silyl; or salts or a derivative thereof.

In some embodiments, the pharmaceutical composition comprises a compound (i.e., isoflavonoid derivative) of formula I:

wherein
R₁, R₃ and R₄ are independently hydrogen, hydroxy, halo, haloalkyl, NR₁₂R₁₃, C₁₋₆ alkoxy, C₁₋₆ fluoroalkyl or C₁₋₆ alkyl;
R₂ and R₅ are independently hydrogen, hydroxy, halo, haloalkyl, NR₁₂R₁₃, C₁₋₆ alkoxy, C₁₋₆ fluoroalkyl or C₁₋₆ alkyl; and
R₆ is hydrogen;
R₇ is hydrogen, haloalkyl, halo or C₁₋₆ alkyl;
R₉ is hydroxy or C₁₋₆ alkoxy;
R₁₀ is hydrogen;
the drawing “ [symbol] ” and R₁₁ together represent a double bond or the drawing “ [symbol] ” represents a single bond and R₁₁ is hydrogen or hydroxy,
R₁₂ and R₁₃ are independently hydrogen, C₁₋₆ alkyl or trialkyl silyl;
or salts or a derivative thereof.

In some embodiments, the pharmaceutical composition comprises a compound (i.e., isoflavonoid derivative) of formula I:

wherein
R₁, R₃ and R₄ are independently hydrogen, hydroxy, halo, haloalkyl, NR₁₂R₁₃, C₁₋₆ alkoxy, C₁₋₆ fluoroalkyl or C₁₋₆ alkyl;
R₂ and R₅ are independently hydrogen, hydroxy, halo, haloalkyl, NR₁₂R₁₃, C₁₋₆ alkoxy, C₁₋₆ fluoroalkyl or C₁₋₆ alkyl; and
R₆ is hydrogen;
R₇ is hydrogen;
R₉ is hydroxy or C₁₋₆ alkoxy;
R₁₀ is hydrogen;
the drawing “ [symbol] ” and R₁₁ together represent a double bond or the drawing “ [symbol] ” represents a single bond and R₁₁ is hydrogen or hydroxy,
R₁₂ and R₁₃ are independently hydrogen, C₁₋₆ alkyl or trialkyl silyl;
or salts or a derivative thereof.

Specific compounds of Formula I, II, or III are shown below:

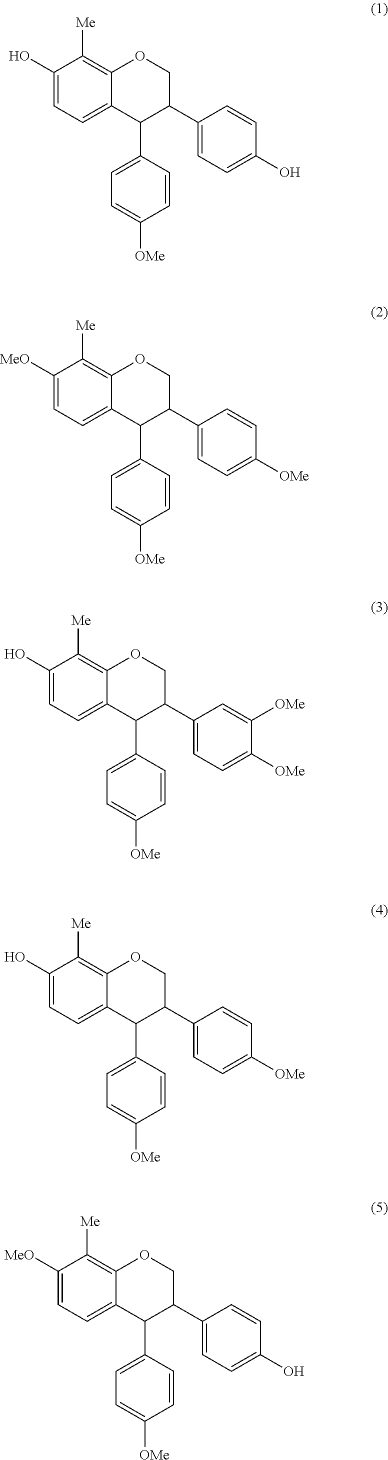

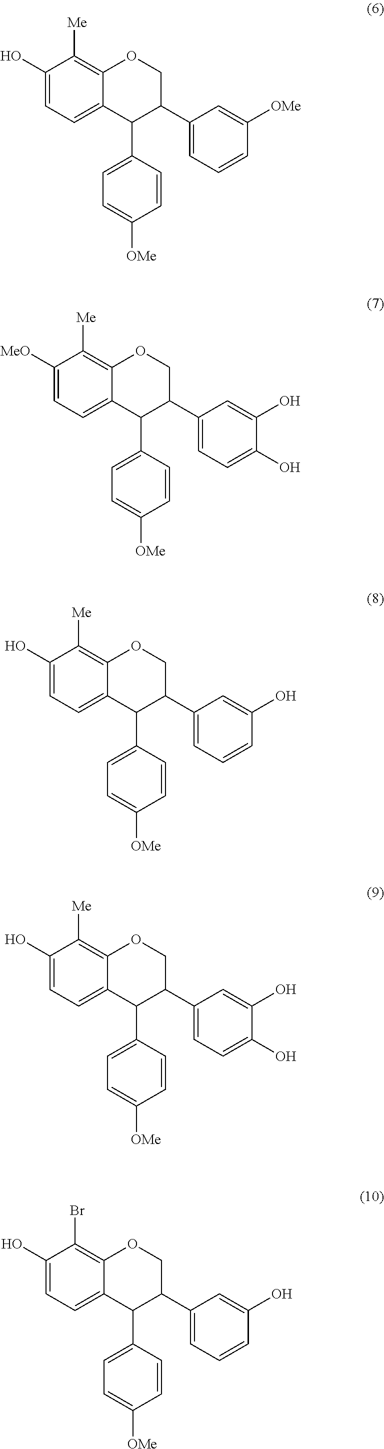

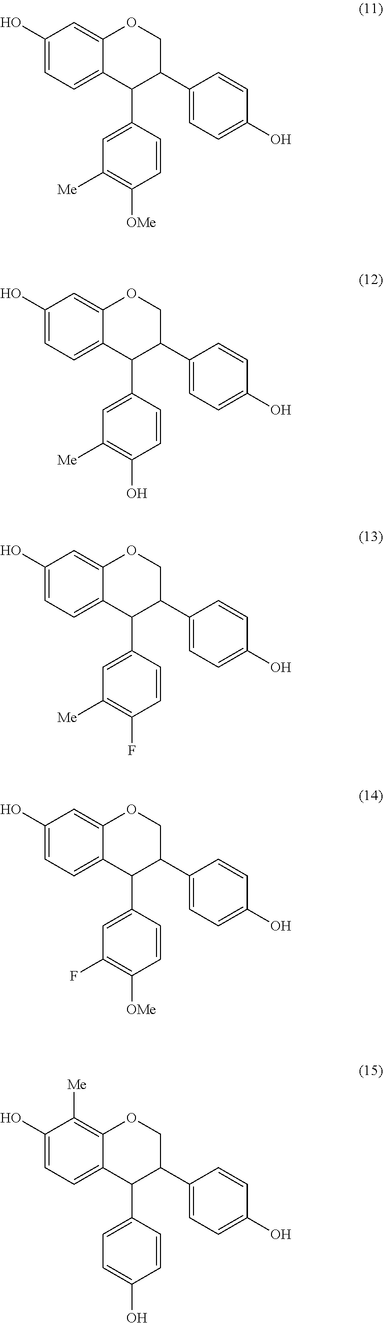

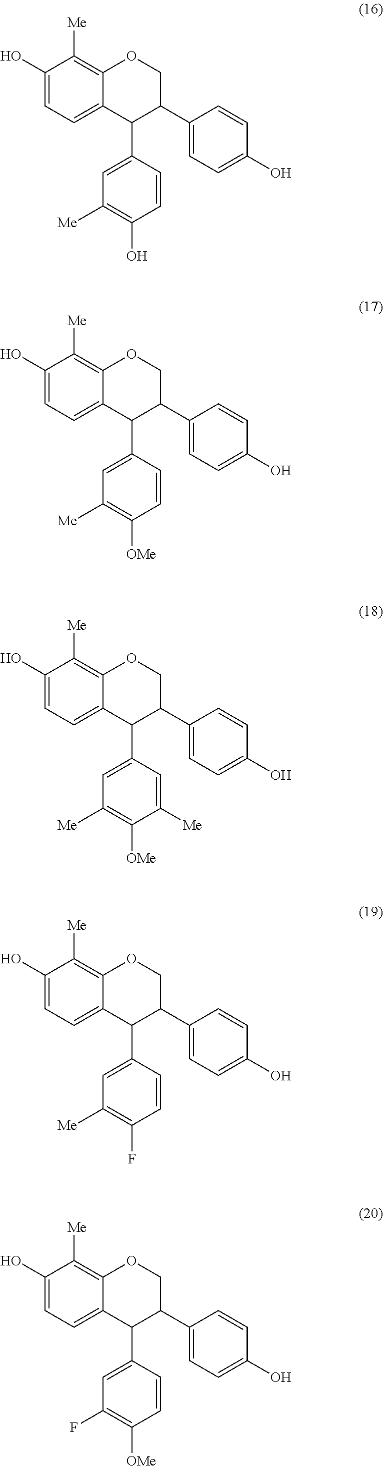

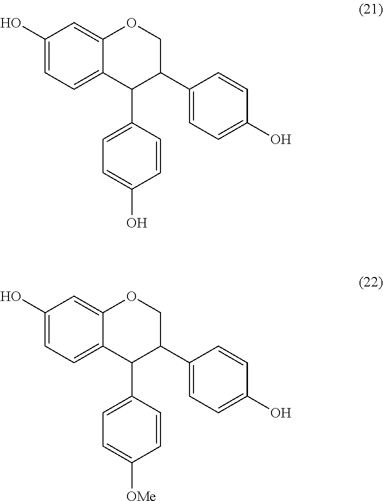

or salts or a derivative thereof.

In specific embodiments, a compound of Formula I, II, or III include:
3-(4-hydroxyphenyl)-4-(4-methoxyphenyl)-8-methylchroman-7-ol (compound 1);
3-(4-methoxyphenyl)-4-(4-methoxyphenyl)-8-methyl-7-methoxychroman (compound 2);
3-(3,4-dimethoxyphenyl)-4-(4-methoxyphenyl)-8-methylchroman-7-ol (compound 3);
3-(4-methoxyphenyl)-4-(4-methoxyphenyl)-8-methylchroman-7-ol (compound 4);
3-(4-hydroxyphenyl)-4-(4-methoxyphenyl)-8-methyl-7-methoxychroman (compound 5);
3-(3-methoxyphenyl)-4-(4-methoxyphenyl)-8-methylchroman-7-ol (compound 6);
3-(3,4-dihydroxyphenyl)-4-(4-methoxyphenyl)-8-methyl-7-methoxychroman (compound 7);
3-(3-hydroxyphenyl)-4-(4-methoxyphenyl)-8-methylchroman-7-ol (compound 8);
3-(3,4-dihydroxyphenyl)-4-(4-methoxyphenyl)-8-methylchroman-7-ol (compound 9);
3-(3-hydroxyphenyl)-4-(4-methoxyphenyl)-8-bromochroman-7-ol (compound 10);
3-(4-hydroxyphenyl)-4-(4-methoxy-3-methylphenyl)chroman-7-ol (compound 11);
3-(4-hydroxyphenyl)-4-(4-hydroxy-3-methylphenyl)chroman-7-ol (compound 12);
3-(4-hydroxyphenyl)-4-(4-fluoro-3-methylphenyl)chroman-7-ol (compound 13);
3-(4-hydroxyphenyl)-4-(4-methoxy-3-fluorophenyl)chroman-7-ol (compound 14);
3-(4-hydroxyphenyl)-4-(4-hydroxyphenyl)-8-methylchroman-7-ol (compound 15);
3-(4-hydroxyphenyl)-4-(4-hydroxy-3-methylphenyl)-8-methylchroman-7-ol (compound 16);
3-(4-hydroxyphenyl)-4-(4-methoxy-3-methylphenyl)-8-methylchroman-7-ol (compound 17);
3-(4-hydroxyphenyl)-4-(4-methoxy-3,5-dimethylphenyl)-8-methylchroman-7-ol (compound 18);
3-(4-hydroxyphenyl)-4-(4-fluoro-3-methylphenyl)-8-methylchroman-7-ol (compound 19);
3-(4-hydroxyphenyl)-4-(4-methoxy-3-fluorophenyl)-8-methylchroman-7-ol (compound 20);
3-(4-hydroxyphenyl)-4-(4-hydroxyphenyl)chroman-7-ol (compound 21); and
3-(4-hydroxyphenyl)-4-(4-methoxyphenyl)chroman-7-ol (compound 22).

It will be clear to persons skilled in the art that in the compounds according to certain embodiments, the aryl substituents on the heterocyclic ring can be cis or trans relative to each other. Preferably in the compounds of Formula I, II, or III according to certain embodiments of the invention, these substituents will be cis.

The compounds of Formula I, II, or III according to some embodiments include two chiral centers. In some embodiments, the compounds of Formula I, II, or III include all the enantiomers and diastereomers as well as mixtures thereof in any proportions. In some embodiments, a compound of Formula I, II, or III also extends to isolated enantiomers or pairs of enantiomers. Some of the compounds herein (including, but not limited to isoflavonoid derivatives and reagents for producing the aforementioned compounds) have asymmetric carbon atoms and can therefore exist as enantiomers or diastereomers. In some embodiments, diastereomeric mixtures are separated into their individual diastereomers on the basis of their physical chemical differences by methods such as chromatography and/or fractional crystallization. In other embodiments, enantiomers are separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

The compounds of Formula I, II, or III according to some embodiments are racemic mixture. In other embodiments, any compound described herein is in the optically pure form (e.g., optically active (+) and (−), (R)- and (S)-, d- and l-, or (D)- and (L)-isomers). In certain preferred embodiments, a compound of Formula I, II, or III is the d-isomer. Accordingly, provided herein, in some embodiments, is the optically active d-isomer having a structure of Formula I, II, or III in enantiomeric excess. In some embodiments, the d-isomer of a compound of Formula I, II, or III is provided in at least 50%, 55%, 60%, 65%, 70%, 75%, 80%, 85%, 88%, 90%, 91%, 92%, 93%, 94%, 95%, 96%, 97%, 98%, 99%, 95%, or 99.9% enantiomeric excess. In other embodiments, the d-isomer of a compound of Formula I, II, or III is provided in greater than 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess. In specific embodiments, of a compound of Formula I, II, or III has greater than 95% enantiomeric excess.

In additional or further embodiments, the compounds described herein are used in the form of pro-drugs. In additional or further embodiments, the compounds described herein are metabolized upon administration to an organism in need to produce a metabolite that is then used to produce a desired effect, including a desired therapeutic effect.

Any compound described herein may be synthesized according to the exemplary syntheses described in U.S. Pat. No. 7,601,855, U.S. Pat. No. 8,080,675, and PCT application number PCT/US2011/058815.

### EXAMPLES

### Example 1

### Studies of the Effect of Compound 1 on Mitochondrial Function in Cancer Stem Cells

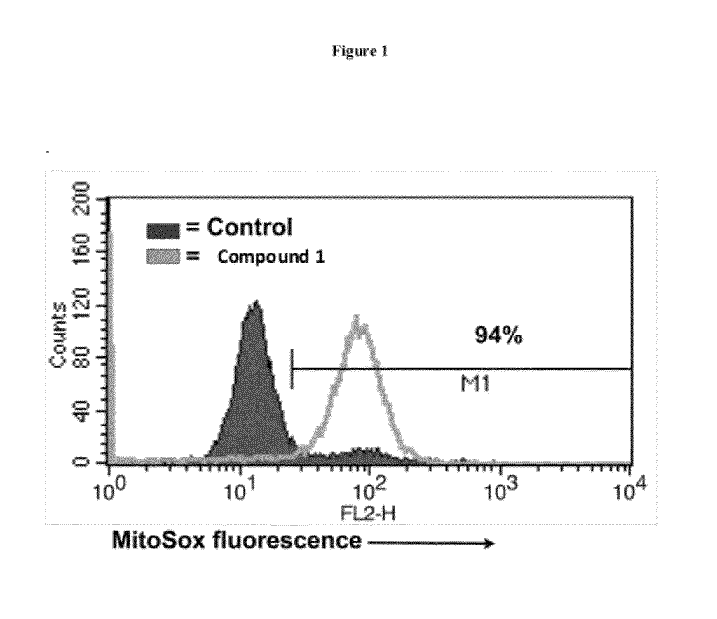

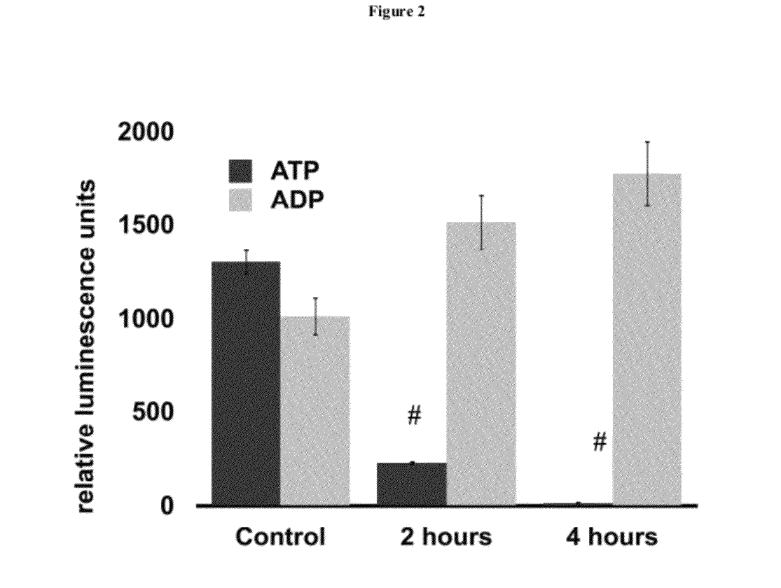

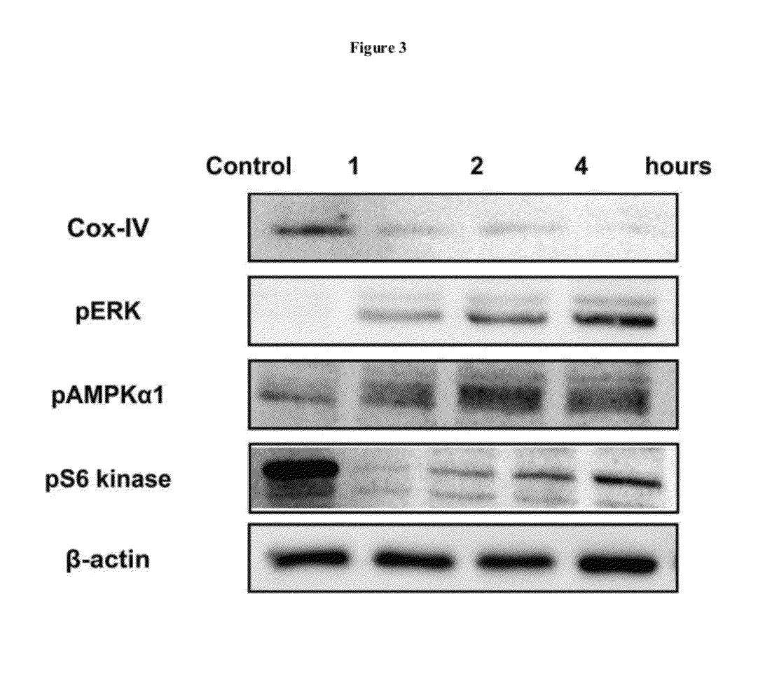

A panel of CD44+/MYD88+ epithelial ovarian cancer (EOC) stem cells was treated with compound 1 (10 μg/mL) at designated times. CD44+/MYD88+ ovarian cancer stem cells are isolated from either tumor tissue or ascites obtained from patients diagnosed with stage III/IV serous ovarian carcinoma. Mitochondrial function was assessed using the JC1 dye (Biovision Inc), MitoSox dye (Invitrogen), and ApoSENSOR ADP/ATP kit (Biovision Inc.). Protein levels were determined using Western blot analysis. Flow cytometry analysis using MitoSox fluorescence indicates that treatment of EOC stem cells with compound 1 resulted in an increase in mitochondrial superoxide (FIG. 1). The impact of compound 1 on energy production was examined by measuring ADP and ATP levels. Analysis using the ApoSENSOR ADP/ATP kit indicated that treatment of EOC stem cells with compound 1 resulted in a decrease in ATP production and accumulated ADP after 2 and 4 hours of treatment (FIG. 2, # p<0.0001 compared to control). The stem cells were treated with increasing with compound 1 (10 μg/mL) at times points shown. Western blot analysis (FIG. 3) indicates that treatment of EOC stem cells with compound 1 decreases activation of Cox-IV and pS6 kinase, and increases activation of ERK and AMPKα1. These results indicates that compound 1 significantly inhibits mitochondrial function in the ovarian cancer stem cell.

### Example 2

### Inhibitory Study of Activity Against Cancer Stem Cells

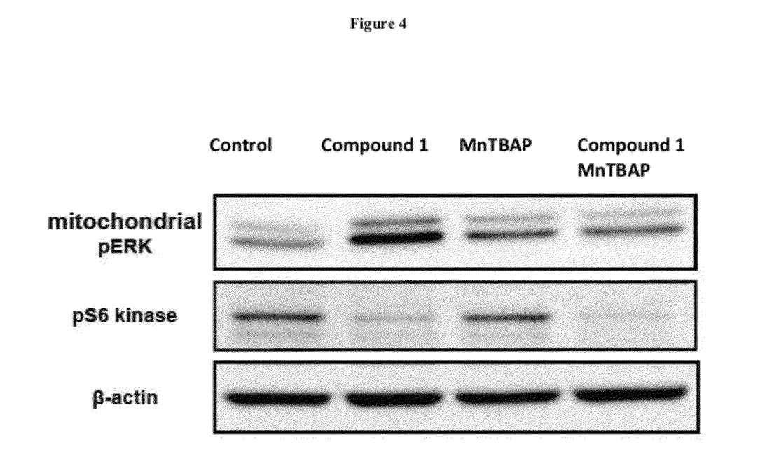

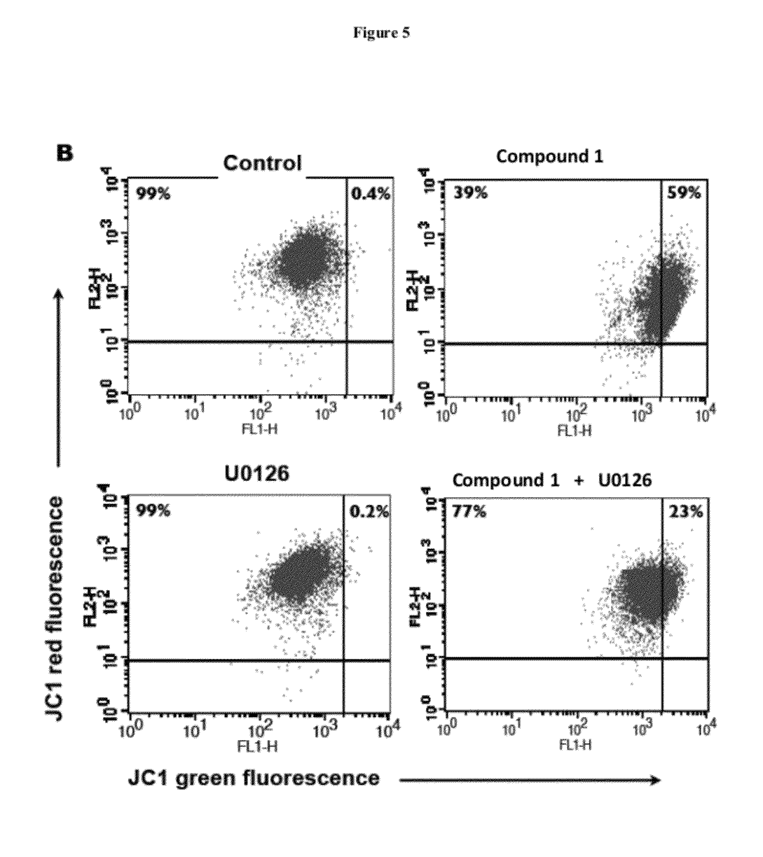

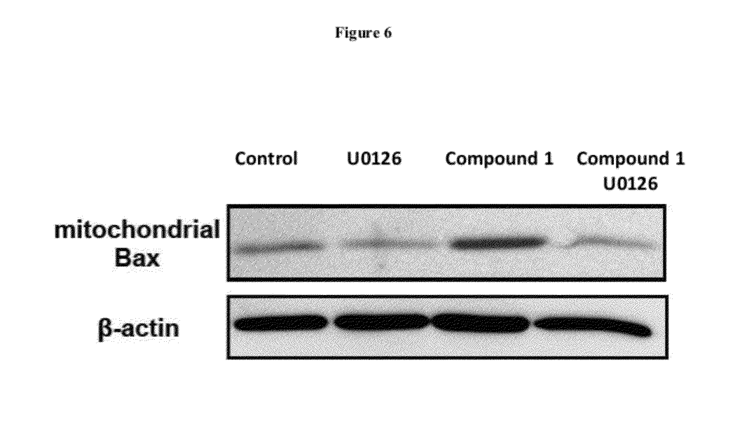

Inhibitory studies were done using the specific MEK inhibitor U0126 (10 μM) or the ROS scavenger, MnTBAP (500 μM). EOC stem cells were treated with compound 1 in the presence or absence of the ROS scavenger, MnTBAP (Alexis Biochemicals). Western blot analysis indicates that MnTBAP inhibited compound 1-induced activation of mitochondrial ERK, but not compound 1-induced decrease in pS6 kinase activity. (FIG. 4) EOC stem cells were treated with compound 1 in the presence or absence of the MEK inhibitor, U0126 (Sigma Aldrich). EOC stem cells were pre-treated with U0126 (10 μM) for 1 hour before treatment with compound 1. Flow cytometry analysis using JC1 green fluorescence illustrates the effect of compound 1 on mitochondrial membrane potential. Pretreatment with U0126 reduced the percentage of cells that lost mitochondrial membrane potential (59% vs 23% for compound 1 along and compound 1 with U0126, respectively) (FIG. 5). The mitochondrial fractions of cells treated with compound 1 in the presence or absence of U0126 were analyzed. Western blot analysis indicates that compound 1 upregulates the proapoptotic Bcl2 family member, Bax. Pre-treatment of the EOC stem cells with U0126 followed by compound 1 prevented compound 1-induced upregulation of mitochondrial Bac. (FIG. 6)

### Example 3

### In Vitro Study of Activity Against Cancer Stem Cells

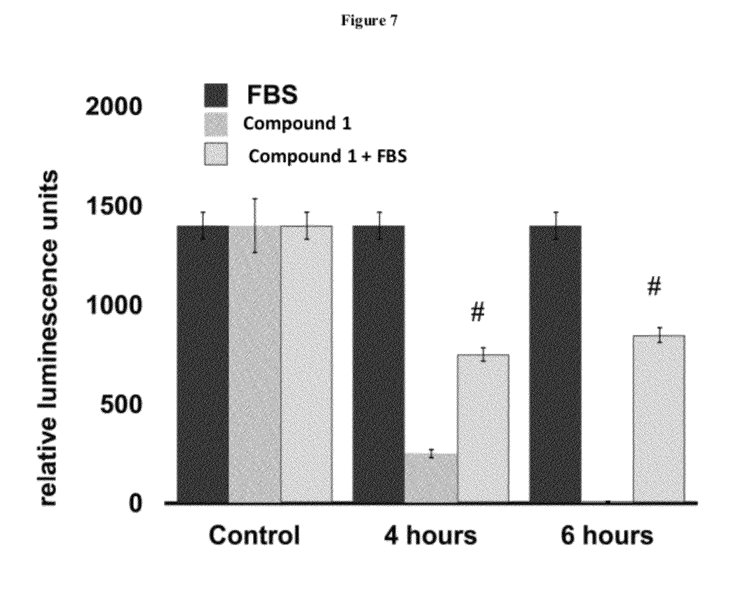

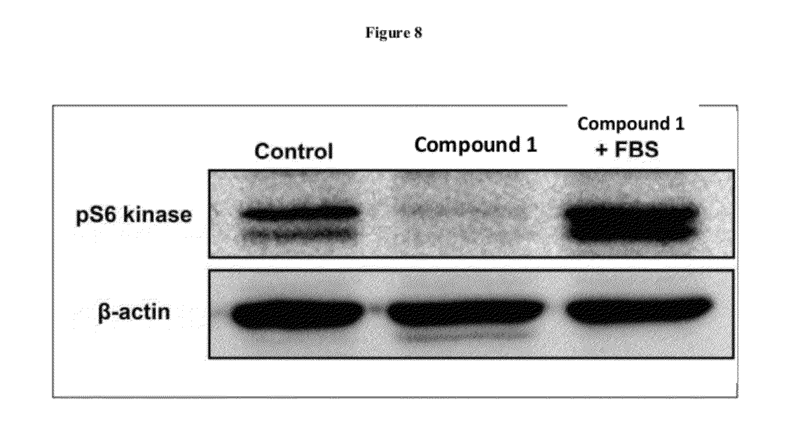

EOC stem cells were treated with compound 1 in the presence of 10% fetal bovine serum (FBS). Abrogation of ATP loss and pS6 kinase inhibition with FBS is indicated by Western blot analysis (FIGS. 7 and 8). Treatment of EOC stem cells with compound 1 in the presence of FBS prevented compound 1-induced decrease in pS6k.

### Example 4

### Treatment for Recurrent Breast Cancer

Phase I/II Human Clinical Trial for the Safety and/or Efficacy of Isoflavonoid for Recurrent Breast Cancer Therapy

Objective: To evaluate the safety and/or efficacy of administered composition comprising compound 1, 12, 15-17, or 21 for treating recurrent breast cancer located on the chestwall following mastectomy.

Study Design: Patients must have recurrent breast cancer. The patients are treated with the composition comprising compound 1, 12, 15-17, or 21 for 6 cycles approximately every 4 weeks. For the first cycle, patients will need to be hospitalized 3 days for measurement of blood levels of drug as well as some additional radiology studies.

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH.

### Example 5

### Treatment for Recurrent Breast Cancer

Phase II Human Clinical Trial for Efficacy of Isoflavonoid for Recurrent Breast Cancer Therapy

Objectives: To determine the efficacy of two different treatment schedules of a composition comprising compound 1, 12, 15-17, or 21 in terms of clinical/radiological response and early progression, in patients with recurrent or metastatic breast cancer; to determine the time to progression and response duration in patients treated with these regimens; to determine the toxic effect of these regimens in these patients; and to correlate molecular markers of mTOR activity in tumor tissue with objective tumor response in patients treated with these regimens.

Study Design: This is a randomized, open label, multicenter study. Patients are stratified according to presence of visceral metastases (yes vs no) and prior chemotherapy regimens for recurrent disease (0 vs 1). Patients are randomized to 1 of 2 treatment arms.

Arm I: Patients receive i.v. compound 1, 12, 15-17, or 21 on days 1 and 15.

Arm II: Patients receive i.v. compound 1, 12, 15-17, or 21 on days 1, 8, 15, and 22. In both arms, courses repeat every 28 days in the absence of disease progression or unacceptable toxicity.

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH.

After completion of study treatment, patients are followed at 4 weeks and then periodically until disease progression.

### Example 6

### Treatment for Breast or Ovarian Cancer in Remission

Phase II Human Clinical Trial for Efficacy of Isoflavonoid for Breast or Ovarian Cancer Remission Therapy

Objectives: To evaluate the efficacy of administered composition comprising compound 1, 12, 15-17, or 21 for treating patients in remission from breast or ovarian cancer; to determine the progression-free survival of cancer patients in remission with this treatment; to determine the toxicity of this treatment in these patients.

Study Design: This is a randomized, open label, multicenter study. Patients must be in remission from histologically confirmed breast or ovarian cancer (i.e., no evidence of disease). Patients must have completed appropriate treatment (e.g., radiotherapy, chemotherapy, and/or surgery) for the primary cancer. Patients must not have received treatment for their cancer within 9 month of beginning the trial.

Patients receive i.v. compound 1, 12, 15-17, or 21 once in months 1, 3, 6, 9, and 12. Treatment continues in the absence of disease progression or unacceptable toxicity. After completion of study treatment, patients are followed every 6 months for 2 years.

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH.

### Example 7

### Treatment for Recurrent Ovarian Cancer

Phase II Human Clinical Trial for Efficacy of Isoflavonoid for Recurrent Ovarian Cancer Therapy

Objectives: To determine the antitumor activity of a composition comprising compound 1, 12, 15-17, or 21 and cisplatin in patients with recurrent platinum-resistant ovarian epithelial or primary peritoneal cancer; to determine the toxicity of this regiment in these patients.

Study Design: This is a non-randomized study. Patients receive the composition comprising compound 1, 12, 15-17, or 21 over 2 hours on days 1-4 and cisplatin IV over 1 hour on days 2 and 3. Courses repeat every 21 days in the absence of disease progression or unacceptable toxicity.

The disease is histologically confirmed as recurrent or persistent ovarian epithelial or primary peritoneal cancer. Patient must have received 1 prior platinum-based chemotherapy regimen (e.g., carboplatin, cisplatin, or other organoplatinum compound) for the primary disease. The initial treatment may have included high-dose, consolidation or extended therapy after surgical or non-surgical assessment. The disease must be considered platinum resistant or refractory according to 1 of the following criteria: (1) treatment-free interval of less than 6 months after platinum-based therapy or (2) disease progression during platinum-based therapy.

Patients should not have had exposure to the compound prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. Patients are followed for 5 years.

1. A method of reducing incidences of cancer recurrence, comprising administering to an individual in cancer remission an effective amount of a compound of formula (I): 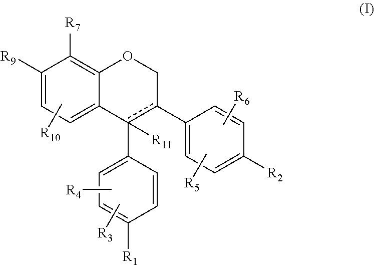
wherein
R₁, R₃ and R₄ are independently hydrogen, hydroxy, halo, haloalkyl, NR₁₂R₁₃, C₁₋₆ alkoxy, C₁₋₆ fluoroalkyl or C₁₋₆ alkyl;
R₂ and R₅ are independently hydrogen, hydroxy, halo, haloalkyl, NR₁₂R₁₃, C₁₋₆ alkoxy, C₁₋₆ fluoroalkyl or C₁₋₆ alkyl;
R₆ is hydrogen;
R₇ is hydrogen, haloalkyl, halo or C₁₋₆ alkyl;
R₉ is hydroxy or C₁₋₆ alkoxy;
R₁₀ is hydrogen;
the drawing “ [symbol] ” and R₁₁ together represent a double bond or the drawing “ [symbol] ” represents a single bond and R₁₁ is hydrogen or hydroxy;
R₁₂ and R₁₃ are independently hydrogen, C₁₋₆ alkyl or trialkyl silyl;
or salts or a derivative thereof; and
wherein the compound of formula (I) reduces cancer cell proliferation.

2. The method of claim 1, wherein the compound of formula (I) is selected from compounds 1 to 22 as set forth below:
3-(4-hydroxyphenyl)-4-(4-methoxyphenyl)-8-methylchroman-7-ol (compound 1);
3-(4-methoxyphenyl)-4-(4-methoxyphenyl)-8-methyl-7-methoxychroman (compound 2);
3-(3,4-dimethoxyphenyl)-4-(4-methoxyphenyl)-8-methylchroman-7-ol (compound 3);
3-(4-methoxyphenyl)-4-(4-methoxyphenyl)-8-methylchroman-7-ol (compound 4);
3-(4-hydroxyphenyl)-4-(4-methoxyphenyl)-8-methyl-7-methoxychroman (compound 5);
3-(3-methoxyphenyl)-4-(4-methoxyphenyl)-8-methylchroman-7-ol (compound 6);
3-(3,4-dihydroxyphenyl)-4-(4-methoxyphenyl)-8-methyl-7-methoxychroman (compound 7);
3-(3-hydroxyphenyl)-4-(4-methoxyphenyl)-8-methylchroman-7-ol (compound 8);
3-(3,4-dihydroxyphenyl)-4-(4-methoxyphenyl)-8-methylchroman-7-ol (compound 9);
3-(3-hydroxyphenyl)-4-(4-methoxyphenyl)-8-bromochroman-7-ol (compound 10);
3-(4-hydroxyphenyl)-4-(4-methoxy-3-methylphenyl)chroman-7-ol (compound 11);
3-(4-hydroxyphenyl)-4-(4-hydroxy-3-methylphenyl)chroman-7-ol (compound 12);
3-(4-hydroxyphenyl)-4-(4-fluoro-3-methylphenyl)chroman-7-ol (compound 13);
3-(4-hydroxyphenyl)-4-(4-methoxy-3-fluorophenyl)chroman-7-ol (compound 14);
3-(4-hydroxyphenyl)-4-(4-hydroxyphenyl)-8-methylchroman-7-ol (compound 15);
3-(4-hydroxyphenyl)-4-(4-hydroxy-3-methylphenyl)-8-methylchroman-7-ol (compound 16);
3-(4-hydroxyphenyl)-4-(4-methoxy-3-methylphenyl)-8-methylchroman-7-ol (compound 17);
3-(4-hydroxyphenyl)-4-(4-methoxy-3,5-dimethylphenyl)-8-methylchroman-7-ol (compound 18);
3-(4-hydroxyphenyl)-4-(4-fluoro-3-methylphenyl)-8-methylchroman-7-ol (compound 19);
3-(4-hydroxyphenyl)-4-(4-methoxy-3-fluorophenyl)-8-methylchroman-7-ol (compound 20);
3-(4-hydroxyphenyl)-4-(4-hydroxyphenyl)chroman-7-ol (compound 21); and
3-(4-hydroxyphenyl)-4-(4-methoxyphenyl)chroman-7-ol (compound 22).

3. The method of claim 1, wherein the individual is in remission from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma.

4. The method of claim 1, wherein the individual is in remission from ovarian cancer or breast cancer.

5. The method of claim 1, further comprising administering to the individual an anti-cancer agent.

6. (canceled)

7. The method of claim 5, wherein the anti-cancer agent is selected from the group consisting of cisplatin, carboplatin, paclitaxel, gemcitabine, doxorubicin, camptothecin, topotecan, and any combinations thereof.

8. The method of claim 1, wherein the combination of the compound of formula (I) and the anti-cancer agent reduces cancer cell proliferation.

9. A method of inhibiting loss of cancer remission comprising contacting an individual having cancer with an effective amount of formula (I): 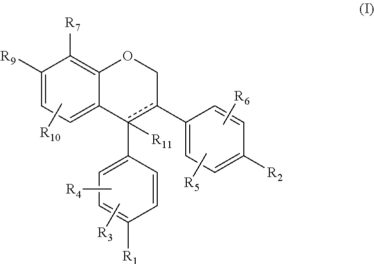
wherein
R₁, R₃ and R₄ are independently hydrogen, hydroxy, halo, haloalkyl, NR₁₂R₁₃, C₁₋₆ alkoxy, C₁₋₆ fluoroalkyl or C₁₋₆ alkyl;
R₂ and R₅ are independently hydrogen, hydroxy, halo, haloalkyl, NR₁₂R₁₃, C₁₋₆ alkoxy, C₁₋₆ fluoroalkyl or C₁₋₆ alkyl;
R₆ is hydrogen;
R₇ is hydrogen, haloalkyl, halo or C₁₋₆ alkyl;
R₉ is hydroxy or C₁₋₆ alkoxy;
R₁₀ is hydrogen;
the drawing “ [symbol] ” and R₁₁ together represent a double bond or the drawing “ [symbol] ” represents a single bond and R₁₁ is hydrogen or hydroxy; and
R₁₂ and R₁₃ are independently hydrogen, C₁₋₆ alkyl or trialkyl silyl;
or salts or a derivative thereof.

10. The method of claim 9, wherein the individual is in remission from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma.

11. The method of claim 9, wherein the individual is in remission from ovarian cancer or breast cancer.

12. The method of claim 9, further comprising administering to the individual an anti-cancer agent.

13. (canceled)

14. The method of claim 12, wherein the anti-cancer agent is selected from the group consisting of cisplatin, carboplatin, paclitaxel, gemcitabine, doxorubicin, camptothecin, topotecan, and any combinations thereof.

15. A method of inducing apoptosis in a cancer-related stem cell, the method comprising contacting said stem cell with a compound of formula (I): 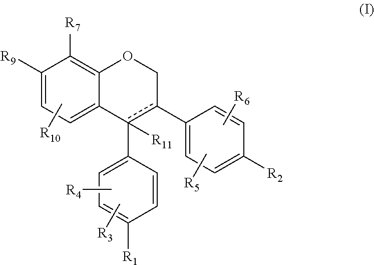
wherein
R₁, R₃ and R₄ are independently hydrogen, hydroxy, halo, haloalkyl, NR₁₂R₁₃, C₁₋₆ alkoxy, C₁₋₆ fluoroalkyl or C₁₋₆ alkyl;
R₂ and R₅ are independently hydrogen, hydroxy, halo, haloalkyl, NR₁₂R₁₃, C₁₋₆ alkoxy, C₁₋₆ fluoroalkyl or C₁₋₆ alkyl;
R₆ is hydrogen;
R₇ is hydrogen, haloalkyl, halo or C₁₋₆ alkyl;
R₉ is hydroxy or C₁₋₆ alkoxy;
R₁₀ is hydrogen;
the drawing “ [symbol] ” and R₁₁ together represent a double bond or the drawing “ [symbol] ” represents a single bond and R₁₁ is hydrogen or hydroxy; and
R₁₂ and R₁₃ are independently hydrogen, C₁₋₆ alkyl or trialkyl silyl;
or salts or a derivative thereof.

16. The method of claim 15, wherein the individual is in remission from bladder cancer, breast cancer, colon cancer, rectal cancer, endometrial cancer, kidney cancer, leukemia, lung cancer, melanoma, ovarian cancer, pancreatic cancer, prostate cancer, cancers of the brain or melanoma.

17. The method of claim 15, wherein the individual is in remission from ovarian cancer or breast cancer.

18. The method of claim 15, further comprising administering to the individual an anti-cancer agent.

19. (canceled)

20. The method of claim 18, wherein the anti-cancer agent is selected from the group consisting of cisplatin, carboplatin, paclitaxel, gemcitabine, doxorubicin, camptothecin, topotecan, and any combinations thereof.

21.-38. (canceled)
